(19) United States
(12) Patent Application Publication
(10) Pub. No.: US 2009/0324746 A1
(43) Pub. Date: Dec. 31, 2009

(54) SYSTEM FOR PRODUCING AND DISPENSING CHLORINE DIOXIDE

(76) Inventors: Andrew M. Bober, Racine, WI (US); Charles Crawford, Racine, WI (US); Dale A. Grinstead, Fairfield, OH (US); Masahiro Nishizawa, Kanagawa-ken (JP); Kenneth J. Roach, Hamilton, OH (US); Carol Anne Rouillard, Loveland, OH (US); James H. Whitehead, Collierville, TN (US); William B. Wright, Norwood, OH (US)

Correspondence Address: JohnsonDiversey, Inc., 8310 16th Street - M/S 509, P. O. Box 902, Sturtevant, WI, 53177-0902, US

(21) Appl. No.: 12/507,720

(22) Filed: Jul. 22, 2009

Related U.S. Application Data
(63) Continuation of application No. 10/709,517, filed on May 11, 2004, now abandoned.
(60) Provisional application No. 60/320,188, filed on May 12, 2003.

(51) Int. Cl. A01N 59/00; A01P 1/00

(52) U.S. Cl. 424/661

(57) ABSTRACT

The invention relates to a multi-component chlorine dioxide producing system. The invention produces effective amounts of chlorine dioxide in five minutes or less without instantaneous production or loss of chlorine dioxide. The invention further includes a dispensing apparatus to allow for the dispensing of the multi component system to allow for insitu production of chlorine dioxide.

### CROSS REFERENCE TO RELATED APPLICATIONS

This application is a continuation of U.S. patent application Ser. No. 10/709,517, filed May 11, 2004, which claims the benefit of U.S. Provisional Patent Application No. 60/320,188, filed May 12, 2003, both of which are incorporated herein by reference in their entirety.

### BACKGROUND OF INVENTION

### Field of Invention

The Invention relates to a process for the production of chlorine dioxide. More specifically the invention relates to a quick and effective method for producing chlorine dioxide without the need for a generator. The invention further relates to a dispensing device capable of holding either a multi compartment bottle or multiple bottles which keep the precursor solutions apart until they are intentionally combined in a controlled manner to produce a solution of chlorine dioxide.

Chlorine dioxide has long been recognized as a preferred biocide. It is well known to be effective against a wide spectrum of organisms. In spite of this, the use of chlorine dioxide as a biocide has heretofore been limited. Chlorine dioxide is a gas and aqueous solutions of it are inherently unstable. Chlorine dioxide readily volatilizes, that is it readily migrates from solution to the gas phase, unless stored in a closed vessel with no headspace. Moreover, chlorine dioxide is subject to photochemical decomposition and subject to chemical decomposition through disproportionation. The net result is that chlorine dioxide solutions have a relatively short shelf life. To compensate for this chlorine dioxide is produced from relatively stable precursor species at the end use facilities. Chlorine dioxide production at end use facilities has heretofore required either a generator to produce chlorine dioxide solutions or a relatively long reaction time to produce chlorine dioxide from the generatorless systems heretofore known.

The generator based systems are systems which use some mechanical or electrical element to facilitate or control the rate of production of chlorine dioxide. Generators fall into two broad categories: chemical and electrochemical. Typically electrochemical generators fall into two categories, those that oxidize a chlorite ion and those that reduce a chlorate ion. All generator based systems produce relatively high concentrations of chlorine dioxide which must be diluted to give use strength solutions. The safety concerns associated with concentrated solutions of chlorine dioxide are well known. Most generators incorporate elaborate safety systems in an attempt to reduce the risk associated with producing, storing and handling these highly concentrated solutions, contributing significantly to the overall cost. The total cost of these generators, including operation and maintenance costs, have limited their application.

Generatorless systems for producing chlorine dioxide are known, however these systems generally require relatively long reaction times (hours) to produce solutions of chlorine dioxide. As with the concentrated chlorine dioxide solutions produced by the generators, these solutions may require dilution to give use strength solutions. The time constraints associated with these systems have limited their application.

Two recent patents have attempted to address the short comings of the current methods of generating chlorine dioxide. However, both of these (Madray, U.S. Pat. No. 6,231,830 and Hei et al., U.S. Pat. No. 6,663,902) require relatively high concentrations of sodium chlorite and other reactants and still require relatively long reaction times.

Madray U.S. Pat. No. 6,231,830 uses an alkali metal chlorite with a alkali metal iodide to produce chlorine dioxide. The patent teaches the need for a buffering agent to maintain the pH above 6.2 and claims a minimum of 300 ppm of the chlorite solution. Madray further requires relatively long reaction times in order to have an effective level of chlorine dioxide produced.

Hei et Al. U.S. Pat. No. 6,663,902 uses an iodo-compound and source of chlorite ions to produce chlorine dioxide. The patent teaches the need for long reaction times and very high levels of chlorite and the iodo-compound in order to have effective amounts of chlorine dioxide produced.

Chlorine dioxide is an excellent sanitizer and disinfectant but without the proper method of production there are just too many limitations to allow for a wide variety of uses. The fact of the matter is, there still remains a need to produce a fast, safe and effective system for the production and use of chlorine dioxide as a disinfectant and sanitizer.

### SUMMARY

The invention discloses a system and formulation that efficiently produces chlorine dioxide in adequate amounts to have the desired biocidal activity while reducing any safety issues and minimizing the loss of chlorine dioxide through volatilization. The invention generally encompasses the use of a chlorite, an activator, a secondary active component and a solvent. In other embodiments of the invention the composition is comprised of a chloride salt, a chlorite and an activator or a chlorite, an activator and a reducing agent to produce the effective amount of chlorine dioxide in less than 5 minutes and preferably less than 3 minutes.

The current invention allows for the production of chlorine dioxide on site which will eliminate any issue as to loss of the chlorine dioxide reducing the effectiveness of the composition and also reduces any volatility and safety issues associated with chlorine dioxide production through most current generators. Further the invention allows for the rapid, safe and efficient production of chlorine dioxide while overcoming the cost and safety issues of the generator systems and the slow production rates and safety issues typically associated with generatorless systems. The invention allows for the reaction time to be reduced to a low level so that the invention can be used without long delays while still not being instantaneous, allowing the invention to be used insitu which eliminates the loss of chlorine dioxide and loss of effectiveness.

In a preferred embodiment, the invention further includes a dispensing apparatus composed of two or more containers or a specialized container with two or more compartments which mixes the components in a predetermined ratio immediately prior to use, thus generating the chlorine dioxide as it is needed. The container or containers of the invention further allows for the storage and mixing of separate components to produce chlorine dioxide as needed while still allowing for the transport and storage of the invention for an extended period of time. The dispensing apparatus includes a support member, at least one connection member connected to the support member, at least one locking member connected to the support member, at least one supply member connected to the connection member, at least one dosing member engaged with at least one supply member and a dispensing member in fluid connection with the dosing member.

### BRIEF DESCRIPTION OF THE DRAWINGS

FIG. 1 is a perspective view of one embodiment of the dispenser and cart.

FIG. 2 is an exploded view of the embodiment shown in FIG. 1.

FIG. 3 is a top view of the embodiment shown in FIG. 1.

FIG. 4 is a back view of the embodiment shown in FIG. 1.

FIG. 5 is an exploded view of a second embodiment.

FIG. 6 is a top view of the second embodiment shown in FIG. 5.

FIG. 7 is a back view of the second embodiment shown in FIG. 5.

### DETAILED DESCRIPTION

The invention comprises a multi component chlorine dioxide producing sanitizing and disinfecting composition which in one embodiment is comprised of a chlorite, an activator, a chloride salt and a solvent. The preferred solvent is water. The invention further includes a diluent, the preferred diluent is water. The activator of the current invention is a component which reduces the pH levels to 5 or lower which aids in the rapid reaction rate in order to produce effective amounts of chlorine dioxide. The preferred embodiment of the activator in the invention is an acid with the most preferred of embodiment being phosphoric acid. The preferred chlorite and chloride are an alkali metal chlorite and an alkali metal chloride respectfully. The most preferred chlorite and chloride are sodium chlorite and sodium chloride respectfully. The chlorite, the activator and the chloride are in amounts which will produce an effective quantity of chlorine dioxide in less than 5 minutes and preferably in less than 3 minutes.

The invention produces effective amounts of chlorine dioxide when the chloride, the chlorite and the activator are present in the preferred amounts as follows; the chloride is less than 3,500 ppm (as sodium chloride), the chlorite is less than 100 ppm (as sodium chlorite) and the activator is less than 5,250 ppm. The invention further produces an effective amount of chlorine dioxide if the chloride to chlorite is in a molar ratio of at least 20:1 respectively.

The invention further requires that certain components must be stored separately in order to prevent the reaction from occurring before desired. The activator and the chlorite must be separated, but the chloride may be combined with either the chlorite or the activator, thus simplifying the system to two components.

Another embodiment of the invention is a multi component chlorine dioxide producing sanitizing and disinfecting composition comprised of a chlorite, an activator and reducing agent. This embodiment also includes a diluent which may be water. The activator of this embodiment may be an acid most preferably phosphoric acid. The chlorite may be an alkali metal chlorite, most preferably sodium chlorite. The invention includes a reducing agent most preferably iodide in amounts which will produce an effective quantity of chlorine dioxide in less than 5 minutes and preferably in less than 3 minutes. The invention may also include one or more surfactants. The preferred surfactants are ones having some biocidal attributes.

Some reducing agents are highly reactive (for example sodium thiosulfate) and readily react with either or both the chlorite and/or the activator. Under these conditions the system must consist of at least 3 separate components. Other reducing agents, for example iodide ion are less reactive with the activator and may be combined with it, thus simplifying the system to two components. In these cases, it may be necessary to add a preservative (for example ascorbic acid) to minimize premature oxidation of the reducing agent by the activator.

The preferred amount of chlorite and reducing agent are less than 300 ppm and 50 ppm, respectively. The pH of the solution is preferably below 5 and most preferable below 2.5. The invention produces effective amounts of chlorine dioxide in less than five minutes and most preferably in less than three minutes. The amount of the activator is less than 1,330 ppm. The ratio of chlorite to reducing agent is less than 50:1. The preferred ratio of chlorite to reducing agent is less than 25:1.

The invention further includes a method of producing chlorine dioxide with a multi component chlorine dioxide producing sanitizing and disinfecting composition where a chlorite, the activator and the reducing agent are included. The activator and the chlorite are stored separately with the reducing agent separate or in with the activator and the components are mixed immediately before use to produce a chlorine dioxide in effective amounts in less than 5 minutes.

Incorporating the activator and the reducing agent in a single component resulted in the reducing agent solution turning dark brown upon standing over an extended period. The preferred stabilizing agent is ascorbic acid. The addition of low levels of a stabilizing agent to the activator and reducing agent delays the onset of the dark color.

The more stabilizing agent present in the activator reducing agent blend, the greater the delay in formation of the dark color. The low levels of the stabilizing agent in the reducing agent/activator blend has minimal, if any, effect on the yield of chlorine dioxide produced from these systems.

One example of the two component product was prepared in which Part A was an aqueous solution of the chlorite and Part B is an activator and reducing agent blend. These components were simultaneously dispensed from separate containers into a water stream via a dual pick-up eductor. Metering tips are used to control the concentrations of Part A and Part B delivered to the water stream.

The wide variability in dilution rates does not affect the yield of chlorine dioxide in terms of absolute concentration at most dilution rates.

### EXAMPLES

The data contained in Tables 1-5 correspond to the system based on chlorite (sodium chlorite), an activator (phosphoric acid) and a chloride salt (sodium chloride).

TABLE 1
% Yield of Chlorine Dioxide vs Molar Ratio of NaCl to NaClO₂ at fixed NaClO₂
concentration of 0.030 gm/l (30 ppm)

| [H₃PO₄] | [NaCl] | [NaClO₂] | | | |
|---|---|---|---|---|---|
| gm/l | gm/l | gm/l | [ClO₂] | molar | yield % |
| (as 75% active | (as 100% | (as 100% | ppm | ratio | based on |
| technical material) | active) | active) | (at 3 minutes) | [NaCl]:[NaClO₂] | stoichiometry |
| 4.31 | 0.00 | 0.030 | 1.0 | 0:1 | 4% |
| 4.31 | 0.74 | 0.030 | 3.0 | 38:1 | 13% |
| 4.31 | 1.10 | 0.030 | 8.0 | 57:1 | 36% |
| 4.31 | 1.45 | 0.030 | 14.6 | 75:1 | 65% |
| 4.31 | 1.74 | 0.030 | 13.7 | 90:1 | 61% |
| 4.31 | 2.26 | 0.030 | 12.8 | 117:1 | 57% |

TABLE 2
% Yield of Chlorine Dioxide vs Molar Ratio of NaCl to NaClO₂ at fixed NaClO₂
concentration of 0.045 gm/l (45 ppm)

| [H₃PO₄] | [NaCl] | [NaClO₂] | | | |
|---|---|---|---|---|---|
| gm/l | gm/l | gm/l | [ClO₂] | molar | yield % |
| (as 75% active | (as 100% | (as 100% | ppm | ratio | based on |
| technical material) | active) | active) | (at 3 minutes) | [NaCl]:[NaClO₂] | stoichiometry |
| 4.31 | 1.10 | 0.045 | 6.4 | 38:1 | 19% |
| 4.31 | 1.45 | 0.045 | 18.7 | 50:1 | 56% |
| 4.31 | 1.74 | 0.045 | 19.3 | 60:1 | 58% |
| 4.31 | 2.26 | 0.045 | 21.8 | 78:1 | 65% |
| 4.31 | 2.96 | 0.045 | 21.4 | 102:1 | 64% |
| 4.31 | 3.48 | 0.045 | 21.1 | 120:1 | 63% |

TABLE 3
% Yield of Chlorine Dioxide vs Molar Ratio of NaCl to NaClO₂ at fixed NaClO₂
concentration of 0.060 gm/l (60 ppm)

| [H₃PO₄] | [NaCl] | [NaClO₂] | | | |
|---|---|---|---|---|---|
| gm/l | gm/l | gm/l | [ClO₂] | molar | yield % |
| (as 75% active | (as 100% | (as 100% | ppm | ratio | based on |
| technical material) | active) | active) | (at 3 minutes) | [NaCl]:[NaClO₂] | stoichiometry |
| 4.31 | 0.00 | 0.060 | 1.3 | 0:1 | 3% |
| 4.31 | 0.37 | 0.060 | 0.8 | 10:1 | 2% |
| 4.31 | 1.45 | 0.060 | 13.0 | 38:1 | 29% |
| 4.31 | 1.91 | 0.060 | 26.0 | 49:1 | 58% |
| 4.31 | 2.26 | 0.060 | 29.4 | 58:1 | 66% |
| 4.31 | 2.96 | 0.060 | 30.0 | 77:1 | 67% |
| 4.31 | 3.48 | 0.060 | 26.0 | 90:1 | 58% |

TABLE 4
% Yield of Chlorine Dioxide vs Molar Ratio of NaCl to NaClO₂ at fixed NaCl
concentration of 1.45 gm/l (1450 ppm)

| [H₃PO₄] | [NaCl] | [NaClO₂] | | | |
|---|---|---|---|---|---|
| gm/l | gm/l | gm/l | [ClO₂] | molar | yield % |
| (as 75% active | (as 100% | (as 100% | ppm | ratio | based on |
| technical material) | active) | active) | (at 3 minutes) | [NaCl]:[NaClO₂] | stoichiometry |
| 4.31 | 1.45 | 0.018 | 6.3 | 125:1 | 47% |
| 4.31 | 1.45 | 0.030 | 14.6 | 75:1 | 65% |
| 4.31 | 1.45 | 0.045 | 18.7 | 50:1 | 56% |
| 4.31 | 1.45 | 0.060 | 13.0 | 38:1 | 29% |
| 4.31 | 1.45 | 0.068 | 10.8 | 33:1 | 21% |
| 4.31 | 1.45 | 0.090 | 5.7 | 25:1 | 9% |
| 4.31 | 1.45 | 0.153 | 0.8 | 15:1 | 1% |

TABLE 5
% Yield of Chlorine Dioxide vs Phosphoric Acid Concentration at fixed NaCl
and NaClO₂ concentrations of 1.45 gm/l (1450 ppm) and 0.045 gm/l (45 ppm)
respectively

| [H₃PO₄] | [NaCl] | [NaClO₂] | | | |
|---|---|---|---|---|---|
| gm/l | gm/l | gm/l | [ClO₂] | Molar | yield % |
| (as 75% active | (as 100% | (as 100% | ppm | Ratio | based on |
| technical material) | active) | active) | (at 3 minutes) | [NaCl]:[NaClO₂] | stoichiometry |
| 0.00 | 1.45 | 0.045 | 0.0 | 50:1 | 0% |
| 0.54 | 1.45 | 0.045 | 10.1 | 50:1 | 31% |
| 1.08 | 1.45 | 0.045 | 15.3 | 50:1 | 46% |
| 2.18 | 1.45 | 0.045 | 14.7 | 50:1 | 44% |
| 3.27 | 1.45 | 0.045 | 16.7 | 50:1 | 50% |
| 4.31 | 1.45 | 0.045 | 18.7 | 50:1 | 56% |
| 5.23 | 1.45 | 0.045 | 18.0 | 50:1 | 54% |

The data contained in Tables 6-10 correspond to the system based on chlorite (sodium chlorite), an activator (phosphoric acid) and a reducing agent (sodium thiosulfate).

TABLE 6
% Yield of Chlorine Dioxide vs Molar Ratio of NaClO₂ to Na₂S₂O₃ at
1.33 gm/l phosphoric acid

| [H₃PO₄] | [Na₂S₂O₃] | [NaClO₂] | | | |
|---|---|---|---|---|---|
| gm/l | gm/l | gm/l | [ClO₂] | Molar | yield % |
| (as 75% active | (as 100% | (as 100% | ppm | Ratio | based on |
| technical material) | active) | active) | (at 3 minutes) | [NaClO₂]:[Na₂S₂O₃] | stoichiometry |
| 1.33 | 0.079 | 0.023 | 0.0 | 0.5:1 | 0% |
| 1.33 | 0.032 | 0.023 | 0.2 | 1.25:1 | 1% |
| 1.33 | 0.016 | 0.023 | 0.0 | 2.5:1 | 0% |
| 1.33 | 0.079 | 0.045 | 0.1 | 1:1 | 0% |
| 1.33 | 0.032 | 0.045 | 3.8 | 2.5:1 | 14% |
| 1.33 | 0.016 | 0.045 | 5.3 | 5:1 | 20% |
| 1.33 | 0.079 | 0.068 | 0.0 | 1.5:1 | 0% |
| 1.33 | 0.032 | 0.068 | 6.8 | 3.75:1 | 17% |
| 1.33 | 0.016 | 0.068 | 7.1 | 7.5:1 | 18% |
| 1.33 | 0.079 | 0.090 | 1.8 | 2:1 | 3% |
| 1.33 | 0.032 | 0.090 | 11.5 | 5:1 | 21% |
| 1.33 | 0.016 | 0.090 | 9.7 | 10:1 | 18% |
| 1.33 | 0.158 | 0.180 | 0.8 | 2:1 | 1% |
| 1.33 | 0.079 | 0.180 | 18.7 | 4:1 | 17% |
| 1.33 | 0.032 | 0.180 | 19.5 | 10:1 | 18% |
| 1.33 | 0.016 | 0.180 | 10.9 | 20:1 | 10% |
| 1.33 | 0.016 | 0.450 | 7.8 | 50:1 | 3% |

TABLE 7
% Yield of Chlorine Dioxide vs Molar Ratio of NaClO₂ to Na₂S₂O₃ at 0.67 gm/l
phosphoric acid

| [H₃PO₄] | [Na₂S₂O₃] | [NaClO₂] | | | |
|---|---|---|---|---|---|
| gm/l | gm/l | gm/l | [ClO₂] | Molar | yield % |
| (as 75% active | (as 100% | (as 100% | ppm | Ratio | based on |
| technical material) | active) | active) | (at 3 minutes) | [NaClO₂]:[Na₂S₂O₃] | stoichiometry |
| 0.67 | 0.079 | 0.023 | 0.0 | 0.5:1 | 0% |
| 0.67 | 0.032 | 0.023 | 0.6 | 1.25:1 | 4% |
| 0.67 | 0.016 | 0.023 | 0.0 | 2.5:1 | 0% |
| 0.67 | 0.079 | 0.045 | 0.1 | 1:1 | 0% |
| 0.67 | 0.032 | 0.045 | 3.1 | 2.5:1 | 11% |
| 0.67 | 0.016 | 0.045 | 4.9 | 5:1 | 18% |
| 0.67 | 0.079 | 0.068 | 0.0 | 1.5:1 | 0% |
| 0.67 | 0.032 | 0.068 | 7.2 | 3.75:1 | 18% |
| 0.67 | 0.016 | 0.068 | 8.4 | 7.5:1 | 21% |
| 0.67 | 0.079 | 0.090 | 3.0 | 2:1 | 6% |
| 0.67 | 0.032 | 0.090 | 11.7 | 5:1 | 22% |
| 0.67 | 0.016 | 0.090 | 9.6 | 10:1 | 18% |
| 0.67 | 0.032 | 0.180 | 21.2 | 10:1 | 20% |

TABLE 8
% Yield of Chlorine Dioxide vs Phosphoric Acid Concentration at a constant
Molar Ratio of 10:1, NaClO₂ to Na₂S₂O₃

| [H₃PO₄] | [Na₂S₂O₃] | [NaClO₂] | | | |
|---|---|---|---|---|---|
| gm/l | gm/l | gm/l | [ClO₂] | Molar | yield % |
| (as 75% active | (as 100% | (as 100% | ppm | Ratio | based on |
| technical material) | active) | active) | (at 3 minutes) | [NaClO₂]:[Na₂S₂O₃] | stoichiometry |
| 1.33 | 0.032 | 0.180 | 19.5 | 10:1 | 18% |
| 0.67 | 0.032 | 0.180 | 21.2 | 10:1 | 20% |
| 0.26 | 0.032 | 0.180 | 17.7 | 10:1 | 16% |
| 0.13 | 0.032 | 0.180 | 13.6 | 10:1 | 13% |
| 0.00 | 0.032 | 0.180 | 0.0 | 10:1 | 0% |

TABLE 9
% Yield of Chlorine Dioxide vs Citric Acid Concentration at a constant Molar
Ratio of 10:1, NaClO₂ to Na₂S₂O₃

| | [Na₂S₂O₃] | [NaClO₂] | | | |
|---|---|---|---|---|---|
| [citric acid] | gm/l | gm/l | [ClO₂] | Molar | yield % |
| gm/l | (as 100% | (as 100% | ppm | Ratio | based on |
| (as 100% active) | active) | active) | (at 3 minutes) | [NaClO₂]:[Na₂S₂O₃] | stoichiometry |
| 1.94 | 0.032 | 0.180 | 14.3 | 10:1 | 13% |
| 0.97 | 0.032 | 0.180 | 13.6 | 10:1 | 13% |
| 0.48 | 0.032 | 0.180 | 13.6 | 10:1 | 13% |
| 0.38 | 0.032 | 0.180 | 10.3 | 10:1 | 10% |
| 0.19 | 0.032 | 0.180 | 8.1 | 10:1 | 8% |

TABLE 10
% Yield of Chlorine Dioxide vs Glycolic Acid Concentration at a constant
Molar Ratio of 10:1, NaClO₂ to Na₂S₂O₃

| | [Na₂S₂O₃] | [NaClO₂] | | | |
|---|---|---|---|---|---|
| [glycolic acid] | gm/l | gm/l | [ClO₂] | Molar | yield % |
| gm/l | (as 100% | (as 100% | ppm | Ratio | based on |
| (as 100% active) | active) | active) | (at 3 minutes) | [NaClO₂]:[Na₂S₂O₃] | stoichiometry |
| 0.38 | 0.032 | 0.180 | 12.8 | 10:1 | 12% |
| 0.19 | 0.032 | 0.180 | 6.3 | 10:1 | 6% |

The data in Tables 11-13 correspond to the system based on chlorite (sodium chlorite), an activator (phosphoric acid) and a reducing agent (potassium iodide).

TABLE 11
% Yield of Chlorine Dioxide vs Molar Ratio of NaClO₂ to KI

| [H₃PO₄] | [KI] | [NaClO₂] | | | |
|---|---|---|---|---|---|
| gm/l | gm/l | gm/l | [ClO₂] | Molar | yield % |
| (as 75% active | (as 100% | (as 100% | ppm | Ratio | based on |
| technical material) | active) | active) | (at 3 minutes) | [NaClO₂]:[KI] | stoichiometry |
| 0.652 | 0.0084 | 0.090 | 9.7 | 20:1 | 18% |
| 0.652 | 0.0167 | 0.090 | 16.8 | 10:1 | 31% |
| 0.652 | 0.0084 | 0.180 | 10.1 | 40:1 | 9% |
| 0.652 | 0.0167 | 0.180 | 18.1 | 20:1 | 17% |
| 0.652 | 0.0334 | 0.180 | 57.1 | 10:1 | 53% |
| 0.326 | 0.0084 | 0.045 | 9.4 | 10:1 | 35% |
| 0.326 | 0.0084 | 0.090 | 10.5 | 20:1 | 19% |
| 0.326 | 0.0167 | 0.180 | 18.7 | 20:1 | 17% |
| 0.260 | 0.0063 | 0.068 | 11.7 | 20:1 | 29% |
| 0.260 | 0.0084 | 0.068 | 12.5 | 15:1 | 31% |
| 0.260 | 0.0084 | 0.090 | 12.2 | 20:1 | 23% |
| 0.163 | 0.0084 | 0.045 | 8.6 | 10:1 | 32% |
| 0.163 | 0.0084 | 0.090 | 11.8 | 20:1 | 22% |
| 0.131 | 0.0084 | 0.068 | 6.8 | 15:1 | 17% |
| 0.131 | 0.0084 | 0.090 | 11.3 | 20:1 | 21% |

TABLE 12
% Yield of Chlorine Dioxide vs Phosphoric Acid Concentration at a constant
Molar Ratio of 20:1, NaClO₂ to KI

| [H₃PO₄] | [KI] | [NaClO₂] | | | |
|---|---|---|---|---|---|
| gm/l | gm/l | gm/l | [ClO₂] | molar | yield % |
| (as 75% active | (as 100% | (as 100% | ppm | ratio | based on |
| technical material) | active) | active) | (at 3 minutes) | [NaClO₂]:[KI] | stoichiometry |
| 0.652 | 0.0084 | 0.090 | 9.7 | 20:1 | 18% |
| 0.326 | 0.0084 | 0.090 | 10.5 | 20:1 | 19% |
| 0.260 | 0.0084 | 0.090 | 12.2 | 20:1 | 23% |
| 0.163 | 0.0084 | 0.090 | 11.8 | 20:1 | 22% |
| 0.131 | 0.0084 | 0.090 | 11.3 | 20:1 | 21% |
| 0.065 | 0.0084 | 0.090 | 16.4 | 20:1 | 30% |
| 0.000 | 0.0084 | 0.090 | 0.0 | 20:1 | 0% |

TABLE 13
Chlorine Dioxide Generation vs Age of Iodide/Acid Blend and Presence of
Ascorbic Acid

| | | | | |
|---|---|---|---|---|
| [H₃PO₄] | | | | |
| gm/l | [KI] | [ascorbic acid] | [NaClO₂] | [ClO₂] |
| (as 75% active | gm/l | gm/l | gm/l | ppm |
| technical material) | (as 100% active) | (as 100% active) | (as 100% active) | (at 3 minutes) |

freshly prepared

| | | | | |
|---|---|---|---|---|
| 0.131 | 0.0084 | 0.00000 | 0.090 | 9.2 |
| 0.131 | 0.0084 | 0.00001 | 0.090 | 9.9 |
| 0.131 | 0.0084 | 0.00010 | 0.090 | 11.0 |
| 0.131 | 0.0084 | 0.00025 | 0.090 | 9.9 |

28 days

| | | | | |
|---|---|---|---|---|
| 0.131 | 0.0084 | 0.00000 | 0.090 | 9.3 |
| 0.131 | 0.0084 | 0.00001 | 0.090 | 9.8 |
| 0.131 | 0.0084 | 0.00010 | 0.090 | 10.1 |
| 0.131 | 0.0084 | 0.00025 | 0.090 | 8.9 |

63 days

| | | | | |
|---|---|---|---|---|
| 0.131 | 0.0084 | 0.00000 | 0.090 | 11.0 |
| 0.131 | 0.0084 | 0.00001 | 0.090 | 11.0 |
| 0.131 | 0.0084 | 0.00010 | 0.090 | 11.0 |
| 0.131 | 0.0084 | 0.00025 | 0.090 | 11.0 |

94 days

| | | | | |
|---|---|---|---|---|
| 0.131 | 0.0084 | 0.00000 | 0.090 | 11.4 |
| 0.131 | 0.0084 | 0.00001 | 0.090 | 11.7 |
| 0.131 | 0.0084 | 0.00010 | 0.090 | 11.7 |
| 0.131 | 0.0084 | 0.00025 | 0.090 | 10.7 |

119 days

| | | | | |
|---|---|---|---|---|
| 0.131 | 0.0084 | 0.00000 | 0.090 | 11.1 |
| 0.131 | 0.0084 | 0.00001 | 0.090 | 11.1 |
| 0.131 | 0.0084 | 0.00010 | 0.090 | 11.4 |
| 0.131 | 0.0084 | 0.00025 | 0.090 | 10.9 |

154 days

| | | | | |
|---|---|---|---|---|
| 0.131 | 0.0084 | 0.00000 | 0.090 | 11.8 |
| 0.131 | 0.0084 | 0.00001 | 0.090 | 11.9 |
| 0.131 | 0.0084 | 0.00010 | 0.090 | 11.0 |
| 0.131 | 0.0084 | 0.00025 | 0.090 | 11.8 |

181 days

| | | | | |
|---|---|---|---|---|
| 0.131 | 0.0084 | 0.00000 | 0.090 | 11.6 |
| 0.131 | 0.0084 | 0.00001 | 0.090 | 11.5 |
| 0.131 | 0.0084 | 0.00010 | 0.090 | 10.5 |
| 0.131 | 0.0084 | 0.00025 | 0.090 | 8.2 |

TABLE 14
Chlorine Dioxide production using a dual pickup eductor to simultaneously
dispense a chlorine (an aqueous sodium chlorite) and activator (an aqueous solution of
phosphoric acid), reducing agent (potassium iodide) and a surfactant (LAS)

| [H₃PO₄] | [LAS] | | | |
|---|---|---|---|---|
| gm/l | gm/l | [KI] | [NaClO₂] | [ClO₂] |
| (as 75% active | (as 98% active | gm/l | gm/l | ppm |
| technical material) | material) | (as 100% active) | (as 100% active) | (at 3 minutes) |
| 0.1416 | 0.1841 | 0.0057 | 0.097 | 13.4 |
| 0.2959 | 0.3846 | 0.0118 | 0.082 | 21.8 |
| 0.2439 | 0.3171 | 0.0098 | 0.097 | 14.1 |
| 0.1779 | 0.2313 | 0.0071 | 0.147 | 13.8 |

TABLE 15
% Yield of Chlorine Dioxide at Differing Ratios of Reactants

| [H₃PO₄] | [LAS] | | | | | |
|---|---|---|---|---|---|---|
| gm/l | gm/l | [KI] | [NaClO₂] | [ClO₂] | | yield |
| (as 75% active | (as 98% | gm/l | gm/l | ppm | Molar | % |
| technical | active | (as 100% | (as 100% | (at 3 | Ratio | based on |
| material) | material) | active) | active) | minutes) | [NaClO₂]:[KI] | stoichiometry |
| 0.1502 | 0.1952 | 0.0060 | 0.113 | 14.9 | 35:1 | 22% |
| 0.1597 | 0.2077 | 0.0064 | 0.108 | 14.7 | 31:1 | 23% |
| 0.1799 | 0.2338 | 0.0072 | 0.099 | 15.5 | 25:1 | 26% |
| 0.2000 | 0.2600 | 0.0080 | 0.090 | 14.9 | 21:1 | 28% |
| 0.2193 | 0.2851 | 0.0088 | 0.081 | 16.4 | 17:1 | 34% |
| 0.2392 | 0.3110 | 0.0096 | 0.072 | 16.0 | 14:1 | 37% |
| 0.2500 | 0.3250 | 0.0100 | 0.068 | 15.7 | 12:1 | 39% |

TABLE 16
Generation of Chlorine Dioxide vs Reaction Time

| | Chloride approach | | | Iodide approach | |
|---|---|---|---|---|---|
| [H₃PO₄] gm/l (as 75% | 4.31 | 4.31 | 4.31 | 0.131 | 0.065 |
| active technical material) | | | | | |
| [NaCl] gm/l | 1.45 | 1.10 | 0.75 | — | — |
| (as 100% active) | | | | | |
| [KI] gm/l | — | — | — | 0.0084 | 0.0042 |
| (as 100% active) | | | | | |
| [NaClO₂] gm/l | 0.030 | 0.030 | 0.030 | 0.090 | 0.090 |
| (as 100% active) | | | | | |
| Molar ratio NaCl:NaClO₂ | 75:1 | 57:1 | 39:1 | | |
| Molar ratio NaClO₂:KI | | | | 20:1 | 40:1 |
| reaction time (sec) | Chlorine Dioxide concentration (ppm) | | | | |
| 0 | 0.0 | 0.0 | 0.0 | 0.0 | 0.0 |
| 30 | 8.7 | 4.9 | 2.2 | 5.8 | 0.9 |
| 60 | 11.4 | 6.0 | 2.7 | 8.7 | 1.8 |
| 90 | | | | 10.3 | 2.5 |
| 120 | 15.0 | 7.0 | 3.1 | 11.2 | 3.1 |
| 150 | | | | 11.7 | 3.6 |
| 180 | 16.6 | 7.3 | 3.2 | 12.0 | 3.9 |
| 210 | | | | 12.1 | 4.3 |
| 240 | 17.6 | 7.4 | 3.3 | 12.2 | 4.6 |
| 270 | | | | 12.3 | 4.8 |
| 300 | 18.2 | 7.5 | 3.3 | 12.3 | 5.0 |

The relatively consistent production of chlorine dioxide, in terms of absolute concentration produced, is surprising and indicates that the method of the present invention is very robust. Thus, the method can withstand relatively large variation in relative dilution ratios of the two components and still produce a consistent concentration of chlorine dioxide.

The data presented clearly indicates that a finite but relatively very short reaction time is required to produce chlorine dioxide in the present method. This is a great benefit. The relatively short reaction time means that the components can be combined by co-eduction or by simultaneously dispensing them together into water which is being or may be directed onto a surface to be treated with chlorine dioxide. The components are mixed and begin reacting as they are being directed onto the surface. Thus chlorine dioxide is generated in situ on the surface to be treated. There is therefore relatively little chlorine dioxide present in the stream as it is directed onto the surface and thus loss of chlorine dioxide to volatilization during spraying becomes virtually a non-issue.

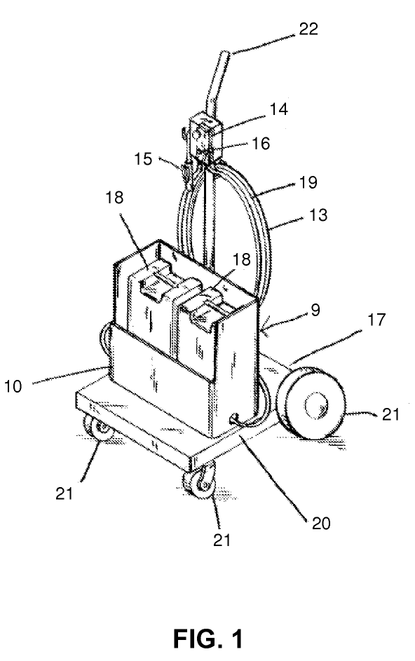

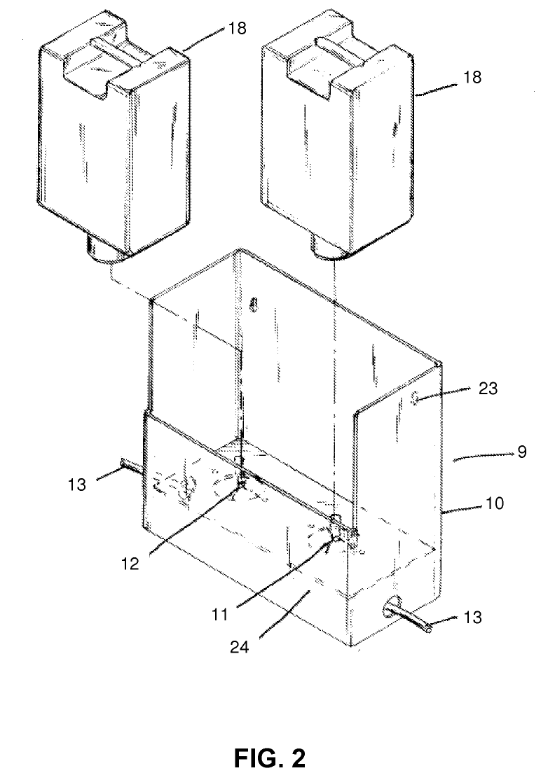

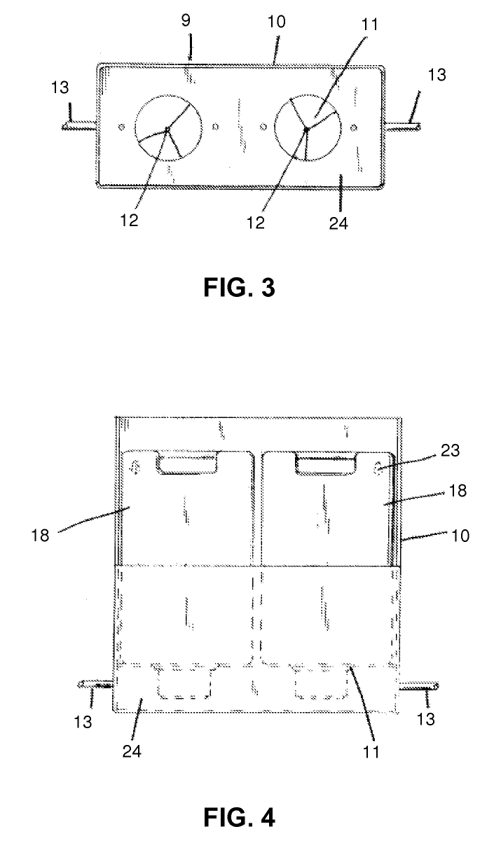

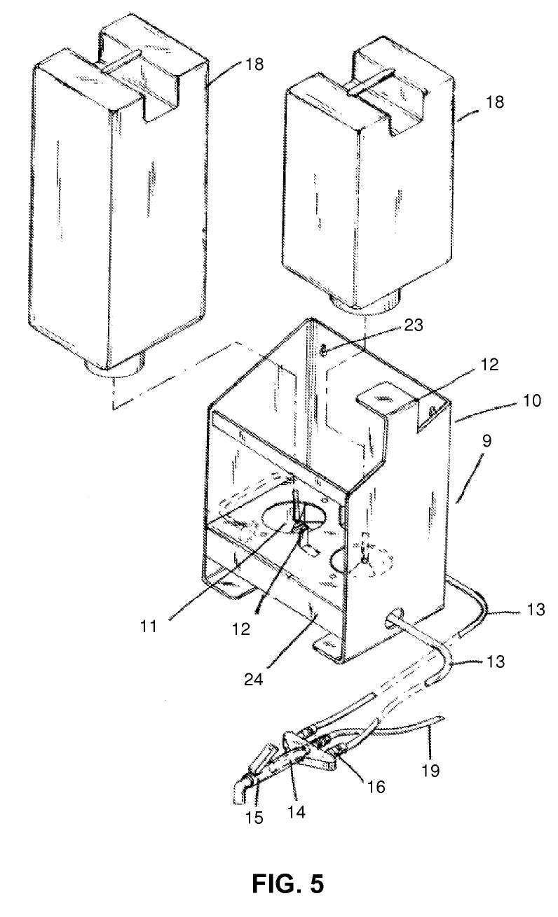

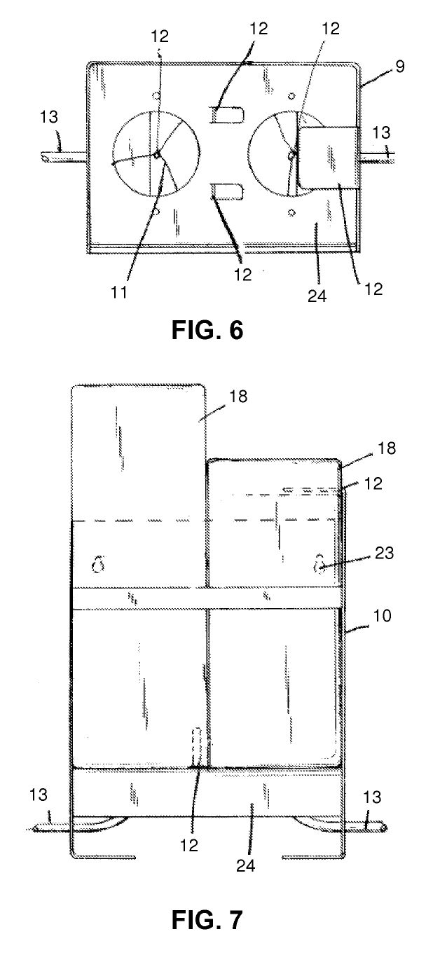

Referring to FIGS. 1-7 the invention further includes a dispensing apparatus generally 9 for combining each component of the multi component chlorine dioxide composition to dispense a single end product. The dispensing apparatus 9 includes a support member 10 supporting a connection member 11 including a locking member 12 with two supply members 13 and a diluent supply member 19 engaged with an dosing member 14 that is connected with a dispensing member 15.

An embodiment of the dispensing apparatus 9 further includes a cart member 17 to allow the dispensing apparatus 9 to be mobile rather than stationary. The cart member 17 includes a platform member 20 and wheels 21 as well as a handle member 22.

Another embodiment of the dispensing apparatus 9 has at least one connection member 11 arranged to engage at least one container member 18 and a locking member 12 to secure at least one container member 18 to the support member 10. The locking member 12 and the connecting member 11 are attached to the support member 10. The locking member 12 secures the container member 18 preventing the container member 18 from disengaging or slipping out of the dispensing apparatus 9. A supply member 13 is attached to at least one container member 18 via the support member 10. The supply member 13 is engaged with at least one metering tip 16 which is engaged with a dosing member 14 in fluid communication with the dispensing member 15. The liquid supply member 19 provides a diluent to the dosing member 14.

The dispensing apparatus 9 has at least one attachment member 23 to secure the dispenser 9 to a surface. The dispensing apparatus 9 may have two or more container members with corresponding connector members 11 that are connected to the support member 10 which is engaged with a base member 24. The dispensing apparatus 9 will have liquid supply members 13 engaged with the base member 24. The liquid supply members are attached to at least one metering tip 16 that connect to at least one dosing member 14 which is in communication with a dispensing member 15.

The invention includes a method for dispensing a multi component composition which includes placing at least one container member 18 in a locking member 12 in the base member 24 in connection to the support member 10 and engaging a connecting member 11 which is connected to a liquid supply member 13 through the support member 10. The liquid supply members 13 transport components of at least one container member 18 to the metering tip 16 which is in fluid communication with the dosing member 14 as well as in connection with the dispensing member 15 to dispense the single end composition product.

The aforementioned embodiments are the preferred embodiments and are not meant in any way to limit the scope of the invention as defined by the appended claims.

What is claimed is:

1. A method of producing a chlorine dioxide sanitizing and disinfecting solution with a multi-component system, the method comprising:
combining a first component comprising chlorite with a second component comprising an activator to produce a solution comprising less than 300 ppm of chlorite; and
forming an amount of chlorine dioxide effective to sanitize and disinfect a surface or a system within five minutes of combining the first and second components, the first and second components of the multi-component system being stored in at least two separate areas.

2. The method of claim 1, wherein the second component additionally comprises a reducing agent.

3. The method of claim 2, wherein the solution comprises less than 50 ppm of reducing agent.

4. The method of claim 1, wherein the pH of the solution is 5 or lower.

5. The method of claim 1, wherein the activator is an acid.

6. The method of claim 5, wherein the activator is phosphoric acid or glycolic acid.

7. The method of claim 1, wherein the chlorite is an alkali metal chlorite.

8. The method of claim 7, wherein the chlorite is sodium chlorite.

9. The method of claim 2, wherein the reducing agent is also antimicrobial in nature.

10. The method of claim 2, wherein the reducing agent is a chloride salt, an iodide salt, or a thiosulfate salt.

11. The method of claim 1, wherein the first component, or the second component, or both further comprise a stabilizing agent.

12. The method of claim 11, wherein the stabilizing agent is ascorbic acid.

13. The method of claim 2, wherein the molar ratio of the chlorite to reducing agent in the solution is less than 50 to 1.

14. The method of claim 1, wherein the first component, or the second component, or both further comprise a surfactant in an amount effective to sanitize and clean a surface or system simultaneously.

15. The method of claim 14, wherein the surfactant has biocidal attributes.

16. The method of claim 1, wherein the solution comprises less than 100 ppm of chlorite.

17. The method of claim 1, wherein forming an amount of chlorine dioxide effective to sanitize and disinfect a surface or a system is done within three minutes.

18. The method of claim 1, wherein greater than 1 ppm of chlorine dioxide is formed.

19. The method of claim 18, wherein greater than 10 ppm of chlorine dioxide is formed.

20. The method of claim 1, wherein the first component and second component are stored in at least two separate areas and the first component and the second component pass through an eductor, wherein the first component and the second component and a diluent stream are combined through at least one metering tip to form a predetermined concentration to be applied to a surface or system.

21. A method of producing chlorine dioxide with a multi-component system having first component and second components, the method comprising:
combining a first component comprising chlorite with a second component comprising an activator and a reducing agent to produce a solution comprising less than 300 ppm of chlorite, less than 50 ppm reducing agent and having a pH less than 5; and
forming greater than 1 ppm of chlorine dioxide within five minutes of combining the first and second components.

22. The method of claim 21, wherein the first and second components of the multi-component system are stored in at least two separate areas.

23. The method of claim 21, wherein the activator is phosphoric acid or glycolic acid.

24. The method of claim 21, wherein the first component, the second component, or both further comprise ascorbic acid.

25. The method of claim 21, wherein greater than 10 ppm of chlorine dioxide is formed.

26. A method of producing chlorine dioxide comprising:
combining a first component comprising chlorite with a second component comprising an activator and a reducing agent, wherein the first component and second component are stored in at least two separate areas and the first component and the second component pass through an eductor, wherein the first component and the second component and a diluent stream are combined through at least one metering tip to make a solution; and
forming an amount of chlorine dioxide effective to sanitize and disinfect a surface or a system within five minutes of combining the first and second components.

27. The method of claim 26, wherein the solution comprises less than 300 ppm chlorite.

28. The method of claim 26, wherein the solution comprises less than 50 ppm of reducing agent.

29. The method of claim 26, wherein greater than 1 ppm of chlorine dioxide is formed.
